(19) United States
(12) Patent Application Publication
(10) Pub. No.: US 2012/0010473 A1
(43) Pub. Date: Jan. 12, 2012

(54) Health and Wellness Tracker

(76) Inventor: Timothy Knuten SULLIVAN, Rumford, RI (US)

(21) Appl. No.: 13/177,272

(22) Filed: Jul. 6, 2011

Related U.S. Application Data
(60) Provisional application No. 61/361,803, filed on Jul. 6, 2010.

(51) Int. Cl. A61B 5/00

(52) U.S. Cl. 600/300

(57) ABSTRACT

A business method utilizing a health and wellness tracker for the optimization of the health program provided for the members of an organization. The present invention provides a wellness tracking service that prompts the members of the organization in predetermined time intervals for health activities. The health of the group of members are compiled into a report and analyzed by the organization for the adjustment and optimization of the health program for the member of the organization.

The current application claims a priority to the U.S. Provisional Patent application Ser. No. 61/361,803 filed on Jul. 6, 2010.

### FIELD OF THE INVENTION

The present invention relates generally to a business process, more specifically, to a business process involving the collection of data regarding the wellness and health of clients.

### BACKGROUND OF THE INVENTION

In most corporations, businesses or organizations, a wellness provider is incorporated to provide health care/wellness services. In large companies, such as those with over 50 employees, health care or wellness providers for employees are required by law. Maintaining or improving aggregate wellness of member populations is a difficult proposition as evidenced by increasing obesity rates and preventable disease rates. Current wellness providers usually do not require employees to make daily reports about diet, exercise and habits. Current wellness providers also do not generate regular unbiased reports based on the health data of its members. Determining the effectiveness of wellness programs is difficult to measure. The present invention is designed to put unbiased, independent, cost effective, actionable data regarding businesses and organizational wellness in the hands of key decision makers. The present invention, by encouraging daily participant entries in the Wellness Tracker, helps participants control their wellness and their ability to modify wellness habits for the healthier, and the business or organization is provided information in a form that allows the businesses or organizations to make better wellness program decisions.

### BRIEF DESCRIPTION OF THE DRAWINGS

FIG. 1 is a diagram of the present invention in the decision cycle for client businesses utilizing the present invention.

### DETAIL DESCRIPTIONS OF THE INVENTION

All illustrations of the drawings are for the purpose of describing selected versions of the present invention and are not intended to limit the scope of the present invention.

The present invention is a business process for data collection of a client's members to gauge and/or track the effectiveness of their wellness program. The present invention is a unique business process that offers a cost effective service to client companies and/or organizations for monitoring the relative health of their member population. The present invention provides an independent and user-friendly tool to monitor the wellness of the members while protecting individual participant information.

The wellness tracker service sends a daily invitation to all participants on a members list provided by the client company, who are then prompted to submit their previous days wellness information. The wellness tracker service provides the participants with the means to enter the wellness information for collection. The wellness information is data regarding each member's health including gender, age, height, weight, hours slept, amount of calories consumed, amount of water consumed, alcohol consumed tobacco usage, cardiovascular exercises performed, number of steps taken, and weight bearing exercises performed. The predetermined time intervals can be daily, weekly, or monthly. The more frequent the data collection, the more accurate the data representing the member population will be. The purpose of collecting the wellness information from the participants is to track the overall improvement or decline of aggregate wellness. Other information that the wellness tracker service is able to collect is the psychological well-being of the members. During the prompting of the members, a series of questions are utilized to gauge the psychological wellbeing of each member. The wellness information of each member is collected to track the relative wellbeing of the group of members over time.

Once the data has been collected for a predetermined length of time, the wellness tracker service will tabulate the wellness information. The tabulated wellness information will be compiled into a wellness report to be sent to the client company. The client company will be able to use the information from the wellness report to adjust the wellness program benefits for its members. In addition to the adjustment of the wellness programs by the client company, members are encouraged to make better health choices. The frequent collection of wellness information is intended to remind members to make healthier choices by discouraging unhealthy activities.

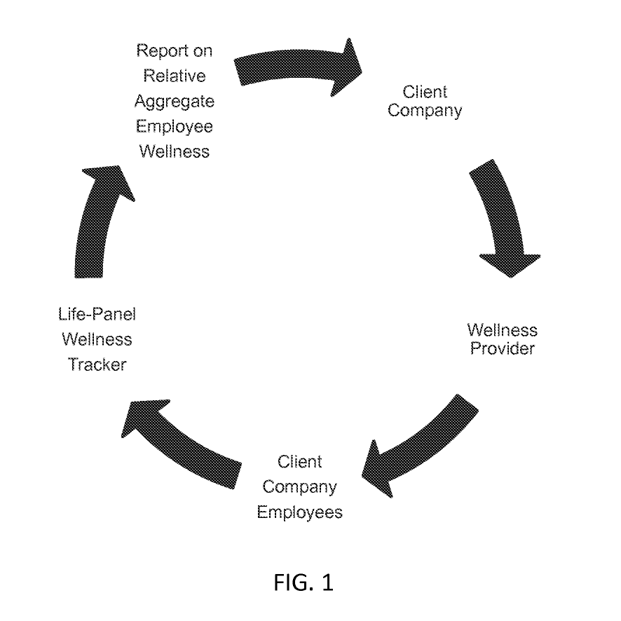

In reference to FIG. 1, the client company is able to provide an optimal program fit for its members through a cycle of information collection and program adjustments. The client company is able to submit their participant list to the wellness tracker service for the collection of the wellness information. The wellness tracker service collects the wellness information and allows the client company to retrieve wellness reports. The client companies are able to utilize the wellness report to adjust and optimize their health program for its members. As differing groups of members may have different conditions, the wellness report can highlight such conditions for each group of members for the modification and optimization of the wellness program.

In an example shown in FIG. 1; a client company partners with a wellness provider to provide optimal healthcare benefits for its employees. The “wellness provider” provides the employees with health services and health benefits. The client company utilizes the wellness tracker to monitor daily health activities of the aggregate employee population. The wellness tracker is able to collect the health information from each participant by means of a regular anonymous periodic health survey. The accumulated data is aggregated and compiled into a report for the client company which can be used to alter services with a wellness provider to ensure optimal health benefits provided. The cycle continues to repeats to continually update and optimize the health benefits for the employees as their health statuses change.

Although the invention has been explained in relation to its preferred embodiment, it is to be understood that many other possible modifications and variations can be made without departing from the spirit and scope of the invention as hereinafter claimed.

What is claimed is:

1. A method for collecting data regarding wellness and health of clients comprises the steps of:
providing a members list, wherein the members list optimally comprises a plurality of participants;
prompting the plurality of participants for wellness information in predetermined time intervals;
providing means to collect the wellness information from the participants;
tabulating of the wellness information; and
compiling of a wellness report from the wellness information.

2. The method for collecting data regarding wellness and health of clients as claimed in claim 1 comprises,
wherein the wellness information includes gender, age, height, weight, hours slept, amount of calories consumed, amount of water consumed, alcohol consumed, tobacco use, cardiovascular exercise performed, number of steps taken, and weigh bearing exercise.

3. The method for collecting data regarding wellness and health of clients as claimed in claim 1 comprises,
wherein prompting the participants includes a series of questions utilized to gauge the psychological wellbeing.

4. A method for a client company to track the health and wellness of members comprises,
submitting a members list to the wellness tracking service; and
receiving of a wellness report from the wellness tracking service.

5. A method for a member of a client company to provide input for wellness programs comprises,
receiving of a anonymous health survey;
responding to the anonymous health survey;
wherein the plurality of wellness data is received by means of email; and
wherein the anonymous health survey is received and responded by means of email.
